(19) United States
(12) Patent Application Publication
(10) Pub. No.: US 2022/0273718 A1
(43) Pub. Date: Sep. 1, 2022

(54) BISPECIFIC ANTIBODY TARGETING OF T REGULATORY CELLS FOR TREATMENT OF INFLAMMATORY CONDITIONS

(71) Applicant: University of Virginia Patent Foundation, Charlottesville, VA (US)

(72) Inventors: Lawrence G. Lum, Charlottesville, VA (US); Archana Thakur, Zion Crosseroads, VA (US)

(21) Appl. No.: 17/632,349

(22) PCT Filed: Aug. 3, 2020
(86) PCT No.: PCT/US2020/044753

§ 371 Date: Feb. 2, 2022

Related U.S. Application Data
(60) Provisional application No. 62/882,455, filed on Aug. 2, 2019.

(51) Int. Cl. A61K 35/17; C07K 16/28; A61P 3/10; A61P 37/06

(52) U.S. Cl. CPC A61K 35/17; C07K 16/2827; C07K 16/2818; A61P 3/10; A61P 37/06; C07K 2317/75

(57) ABSTRACT

Provided are methods and compositions for treating disease and/or disorders associated with inflammatory conditions, such as autoimmune diseases, graft-vs-host diseases, and/or organ graft rejection (collectively AGO), In some embodiments, the methods include isolating peripheral blood mononuclear cells from a patient suffering from AGO, arming a population of T_{REGS} with a bispecific antibody directed at T_{REG} cells and at target antigens on pancreatic islet cells or other AGO inflamed target tissues under conditions, wherein generation of T_{REGS}, bispecific antibody is used to arm A_{TREGS} and target cells expressing autoimmune antigens, AT_{REG} cells binding to target cells expressing autoimmune antigens, suppression of inflammatory activity by immune cells in the tissue microenvironment by AT_{REGS}, and infusing a composition comprising the A_{TREGS} armed with a bispecific antibody into the subject to thereby treat the AGO in the patient. Also provided are compositions that include AT_{REGS} targeting check-point antigens on T cells and/or autoimmune antigen targets in inflamed tissue of subjects.

### CROSS REFERENCE TO RELATED APPLICATION

The presently disclosed subject matter claims the benefit of U.S. Provisional Patent Application Ser. No. 62/882,455, filed Aug. 2, 2019; the disclosure of which is incorporated herein by reference in its entirety.

### TECHNICAL FIELD

The presently disclosed subject matter relates in some embodiments to methods for treating diseases and disorders associated with inflammation in a subject, such as but not limited to autoimmune diseases, graft-vs-host diseases, and/or organ graft rejection (collectively AGO), with TREGS armed with bispecific antibodies directed at TREG cells and/or target antigens on pancreatic islet cells and/or other AGO inflamed target tissues under conditions. Also provided are compositions that include AT_{REGs} targeting checkpoint antigens on T cells and/or autoimmune antigen targets in inflamed tissue of subjects.

### BACKGROUND

According to the American Diabetes Association, in the next 48 hours, 7,670 Americans will be diagnosed with diabetes (one person every 23 seconds) at an annual cost of ˜$245 billion, with 43% of the costs used for emergency care. Type 1 diabetes (T1D) is the harshest form of this disease, typically beginning at an early age and affecting millions of children and adults. T1D has a strong genetic component and is characterized by autoimmune destruction of insulin-producing beta (β) cells in the pancreas, resulting in uncontrolled blood sugar levels. The only proven treatments of T1D are multiple daily insulin injections, technology-based continuous insulin infusions, and whole-organ pancreas transplantation. External insulin replacement is not nearly as efficient as natural secretion of insulin. T1D patients experience life-threatening events such as severe hypoglycemia or diabetic ketoacidosis, and long-term complications such as nephropathy, neuropathy with amputations, and retinopathy leading to blindness are common despite improved glycemic control (Lind et al., N Engl J Med 2014; 371:1972-82).

Modulation of the immune system with drugs or adoptive cell transfer of TREGS have altered the insulin requirement, but the effects have not been sustainable. Improved delivery and function of TREGS represents a need in the art, for example, to test whether TREGS are clinically effective (Bluestone et al., Sci Transl Med 2015; 7:315; Keymeulen et al., Diabetologia 2010; 53:614-23. Epub 2010 Jan. 14; Gottlieb et al., Diabetes Care 2010; 33:826-32; Epub 2010 Jan. 12; Herold et al., N Engl J Med 2002; 346:1692-8; Moran et al., Lancet 2013; 381:10.1016/S0140-6736(13)60023-9. Epub 2013 Apr. 5; Orban et al., Lancet 2011; 378:412-9; Epub 2011 Jun. 28; Pescovitz et al., N Engl J Med 2009; 361:2143-52; Mastrandrea et al., Diabetes Care 2009; 32:1244-9. Epub 2009 Apr. 14; Long et al., Diabetes 2012; 61:2340-8; Epub 2012 Jun. 20.

It is known that pre-T1D and T1D patients develop autoantibodies to the pancreatic β cell antigens IA2, GAD65, ZNT8, and [pro-insulin] that predict the development of T1D. However, therapeutic approaches for T1D and other autoimmune diseases represent a continuing need in the art.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In accordance with some embodiments, the presently disclosed subject matter provides a method of treating a disease and/or disorder associated with inflammation. In some embodiments, the method comprises administering to the subject an effective amount of a composition comprising a targeted activated regulatory T cell (AT_{REG}), which selectively binds a cell associated with disease and/or disorder associated with inflammation in the subject, to thereby treat the disease and/or disorder associated with inflammation in the subject. In some embodiments, the targeted activated regulatory T cell (AT_{REG}) is a bispecific antibody (BiAb) armed activated regulatory T cell (AT_{REG}). In some embodiments, the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof.

In some embodiments, the method comprises (a) isolating peripheral blood mononuclear cells from a subject suffering from a disease and/or disorder associated with inflammation; (b) arming a population of T regulatory cells (T_{REG}s) with a bispecific antibody (BiAb) directed at a TREG cell and to target an antigen of a cell associated with the disease and/or disorder associated with inflammation in the subject, optionally pancreatic islet cells or other inflamed target cell, under conditions wherein: (i) generation of T regulatory cells (T_{REG}s) occurs; (ii) bispecific antibody are used to arm TREG cells (AT_{REG}s) and target the cell having the antigen, optionally an autoimmune antigen (such as IA2, GAD65, ZNT8, pro-insulin, or other autoantigen) or an infectious agent antigen (such as a COVID19 antigen); (iii) AT_{REG}s bind to the cell; and (iv) suppression of inflammatory activity by immune cells in the subject by AT_{REG} occurs; and (c) infusing a composition comprising the AT_{REG}s armed with a bispecific antibody into the subject, thereby treating the subject.

In some embodiments, the method comprises infusing AT_{REG}s intravenously and/or directly injecting AT_{REG}s into an affected organ and/or site with or without an additional therapeutic agent, optionally IL-2, immune suppressive cytokines, immunosuppressive agents, and/or immunosuppressive monoclonal antibodies. In some embodiments, the AT_{REG}s have been induced by ex vivo stimulation with an anti-CD3 Mab/IL-2 and/or anti-CD3/anti-CD28 in combination with rapamycin and/or temsirolimus with TGF-beta, optionally in a range of 1 ng/ml to 200 ng/ml. In some embodiments, the T_{REG}s have been induced and/or maintained by an ex vivo treatment or by an in vivo treatment of the subject with a checkpoint inhibitor antibody selected from the group consisting of anti-PD1 (CD279), anti-PDL1 (CD274 or B6 B7-H1), anti-PDL2 (CD273), anti-CTLA4 (CD152), or any combination thereof, wherein the checkpoint inhibitor antibody or antibodies enhance suppressor activity in the T_{REG}s. In some embodiments, the potency and phenotype of TREGS can be induced, enhanced, and/or maintained by in vitro arming or in vivo arming the T_{Reg}s with a BiAb with an anti-T cell partner being a checkpoint inhibitor agonistic to induce suppressor activity. In some embodiments, the AT_{REG}s are from an autologous donor to the patient and/or are from an allogeneic donor to the patient. In some embodiments, the T_{REG}s is a CD4⁺/FoxP3⁺ cell or a CD8⁺/FoxP3⁺ cell.

In some embodiments, arming doses provide 50% suppression at E:T of 1:1 to 5:1 in an immune suppression assay. In some embodiments, the T_{REG}s can be armed with BiAbs doses ranging from 0.01 ng/million to 500 ng/million T_{REG}s.

In some embodiments, the BiAb comprises two monoclonal antibodies. In some embodiments, the BiAb is directed at any non-activating T cell antigen. In some embodiments, the AT_{REG} is targeted at any surface antigen on pancreatic islet or organ cell being damaged by an inflammatory process, optionally IA2, GAD65, or ZNT8, or is targeted at a COVID 19 antigen, optionally a SAR-CoV2 antigen, further optionally spike, S1 receptor binding domain, nucleocapsid, or membrane antigen.

In some embodiments, the presently disclosed subject matter provides a composition comprising an effective amount of a targeted activated regulatory T cell (AT_{REG}), which selectively binds a cell associated with disease and/or disorder associated with inflammation in the subject; and a pharmaceutically acceptable carrier. In some embodiments, the targeted activated regulatory T cell (AT_{REG}) is a bispecific antibody (BiAb) armed activated regulatory T cell (AT_{REG}). In some embodiments, the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof. In some embodiments, the composition is for use in treating a disease and/or disorder associated with inflammation in the subject.

In some embodiments, the presently disclosed subject matter provides for the use of a composition comprising a targeted activated regulatory T cell (AT_{REG}), which selectively binds a cell associated with disease and/or disorder associated with inflammation in the subject, to treat the disease and/or disorder associated with inflammation in the subject and/or to prepare a medicament for treating a disease and/or disorder associated with inflammation in the subject. In some embodiments, the targeted activated regulatory T cell (AT_{REG}) is a bispecific antibody (BiAb) armed activated regulatory T cell (AT_{REG}). In some embodiments, the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof.

In some embodiments, a composition for treating a mammal suffering from autoimmune diseases, graft-vs-host diseases, and/or organ graft rejection (collectively AGO) is disclosed. In some embodiments, the composition comprises AT_{REG}s targeting checkpoint antigens on the mammal's T cells and autoimmune antigen targets in inflamed tissue of the mammal.

Accordingly, it is an object of the presently disclosed subject matter to provide compositions and methods for treating diseases or disorders characterized by inflammatory conditions. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIG. 1 is a schematic diagram showing a strategy for arming T_{REGs} with bispecific antibodies to PD1, PDL1, or CD2 directed at the T cell and anti-IA2 or anti-GAD65 directed at the pancreatic islet cells.

FIG. 2 is a schematic diagram showing immuno-pathophysiology of T1D. Beta cell damaged triggering immunization of immune system that produces autoreactive T cells and autoantibodies directed at IA2, GAD65, ZNT8 and insulin. The ability of T_{REGs} to suppress the pathways is shown.

FIGS. 3A and 3B are each a plot showing that T_{REGs} induced with soluble OKT3. Increased expression of FoxP3 after 13 days of in vitro expansion from whole PBMC. (FIG. 3A) Representative example of total CD4 plot is shown. (FIG. 3B) Representative example of CD25+ FoxP3 (gated on CD4) plot is shown.

FIG. 4 is a set of bar graphs showing that human peripheral mononuclear lymphocytes (PBL) were stimulated with either PW or OKT3 for T cell dependent IgG synthesis. Top left panel shows the inhibition of IgG synthesis by inducible T regulatory cells (T_{REGs}) from a single individual produced by different induction conditions (T_{REGs} 1-4). Top right panel shows suppression of OKT3 stimulated IgG synthesis by T_{REGs} added at different ratios. Lower panel shows percent suppression of PW stimulated (lower left) or OKT3 stimulated IgG synthesis by T_{REGs}.

FIG. 5 is a graph showing the kinetics of anti-IA2 ab synthesis and suppression by T_{REGs}. Specific IgG anti-IA2 B cell Eli Spots produced by PBMC from a normal subject were determined at 7, 14, and 19 days of culture at IA2 dose 2.0 ug/ml. T_{REGs} (5×10⁵) were added to 2.0×10⁶ PBMC per well. IgG anti-IA2 B cell EliSpots (spots/10⁶ cells cultured).

FIG. 6 is a set of graphs showing that T cells manufactured in the TREG condition suppress inflammatory cytokine secretion by effector Th1/Tc1 cells. A steady-state apheresis sample was enriched for lymphocytes and plated into a G-Rex culture vessel, and incubated in complete media containing mTOR inhibitors and exogenous cytokines for ex vivo manufacture of TREG cells. In parallel, T cells were cultured in the presence of the type I polarizing cytokine IFN-α to generate effector Th1/Tc1 cells. After ex vivo culture, the Th1/Tc1 cells were co-stimulated with anti-CD3/anti-CD28 beads either alone or in combination with delayed addition (after 24 hrs) of co-stimulated TREG cells at a TREG cell-to-effector Th1/Tc1 cell ratio of 1:1 or 1:3. After 24 hours, the supernatants were tested for content of IFN-γ, GM-CSF, and TNF-α.

FIG. 7 is a graph showing suppression of TT-induced specific Ab synthesis by T_{REGs}. T_{REGs} (5 or 10×10⁵) added to 200 μl cultures of 2.0×10⁶ normal PBMC per well. IgG anti-TT specific B cell EliSpots reported as specific anti-TT B cells/10⁶ cells cultured.

FIG. 8 is a set of images showing binding of T_{REG}s armed with anti-PD1×anti-IA2 to islets. T_{REG}s armed with 500 ng of anti-PD1×anti-IA2 BiAb/10⁶ cells were mixed with freshly isolated islets at a ratio of 25:1 and incubated at 37° C. for 4 hours.

### DETAILED DESCRIPTION

Headings are included herein for reference and to aid in locating certain sections. These headings are not intended to limit the scope of the concepts described therein under, and these concepts can have applicability in other sections throughout the entire specification.

The presently disclosed subject matter pertains in some embodiments to an IA2 antigen-induced in vitro primary specific antibody (Ab) synthesis assay to measure specific anti-islet cell antigen IA2 antibody synthesis by normal donor peripheral blood mononuclear cells (PBMC). This assay, which uses IA2 antigen to stimulate in vitro primary specific anti-IA2 IgM and IgG Ab synthesis as measured by B cell enzyme-linked immune absorbent spot (ELISpot) assays, assesses whether regulatory T cells (T_{REGs}) can suppress specific anti-IA2 Ab synthesis. Methods have been developed to generate polyclonal natural and induced T_{REGs} cells. Several antigen-specific and non-specific assays were used, re-purposed, or newly developed to validate the function of T_{REGs}. One assay embodiment is a transwell system that separates inflammatory Th1 cells from the TREG, thereby evaluating T_{REGs} suppression of Th1 activated inflammatory cytokines in an antigen-independent manner. The ability of T_{REGs} to suppress pokeweed mitogen (PWM) activated in vitro polyclonal immunoglobulin (Ig) synthesis is also examined.

The presently disclosed subject matter relates in some embodiments to immunotherapy approaches employing bispecific antibody-armed T cells (BATs). A patient's T cells are isolated and cultured ex vivo with a bispecific antibody (e.g., two antibodies conjugated via a linker), and then injected back into the patient. In some embodiments, the presently disclosed subject matter activates regulatory T cells (T_{REGs}) with bispecific antibodies for use against autoimmune diseases, such as but not limited to, diabetes.

A non-limiting example of AGO and/or other diseases and/or disorders associated with inflammation is Type 1 diabetes (T1D). The ability of TREG cell therapy to induce tolerance in Type 1 diabetes (T1D) patients may be enhanced if such TREG cells were targeted to the site of inflammation. To address this, the presently disclosed subject matter relates to adapt bispecific antibody (BiAb) targeting of T_{REGs} to improve delivery of natural and induced polyclonal T_{REGs} to sites of inflammation including, but not limited to, the islet cell microenvironment. As such, the presently disclosed subject matter relates in some embodiments to novel methods to generate potent polyclonal T_{REGs}, new assays to validate the function of the T_{REGs}, and BiAb-armed T_{REGs} traffic to inflammatory sites.

In some embodiments, BiAb armed TREG cells target COVID 19 antigens, such as SAR-CoV2 antigens, such as spike, S1 receptor binding domain, nucleiocapside, and membrane antigens, on infected cells and suppress the inflammatory and cytokine release syndrome seen in the inflammatory acute respiratory distress syndrome. In some embodiments, universal allogeneic donor cells are used as the primary donor. In some embodiments, donor cells are autologous cells, which in some embodiments, are produced in 6 days. In some embodiments, a therapeutic composition in accordance with the presently disclosed subject matter is provided as an “off-the shelf” product, such as frozen T_{REGs} armed with anti-PD1×anti-spike, anti-PD1×anti-nucleocapsid, and the like. The T cell targeting antibody can be any non-activating anti-T cell antibody, such as CD2, CD45, etc. used to armed TREG. Zhao et al., J Appl Physiol 104:1793-1800, 2008; Sen et al., J Hematother Stem Cell Res 2001 10:247-60; Lum et al., Clinical Breast Cancer, Vol. 4, No. 3, 212-217, 2003; Lum, Expert Opin. Drug Discov. (2008) 3(9):1-17; Lum et al., Biol Blood Marrow Transplant 18: 1012-1022 (2012); Lum et al., Biol Blood Marrow Transplant 19 (2013) 925-933.

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, a subject is a human.

Similarly, all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, cancer. The additional compounds can also be used to treat symptoms associated with the injury, disease, or disorder, including, but not limited to, pain and inflammation.

The term “adult” as used herein, is meant to refer to any non-embryonic or non-juvenile subject.

A disease or disorder is “alleviated” if the severity of a symptom of the disease, condition, or disorder, or the frequency with which such a symptom is experienced by a subject, or both, are reduced.

“Allogeneic” refers to cells or to a graft derived from a different animal of the same species.

As used herein, amino acids are represented by the full name thereof, by the three letter code corresponding thereto, or by the one-letter code corresponding thereto, as indicated in Table 1:

TABLE 1
Amino Acid Codes and Functionally Equivalent
Codons

| | 3- | 1- | Functionally |
|---|---|---|---|
| | Letter | Letter | Equivalent |
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an amino acid in which the side chain is fused to the amino group.

Amino acids have the following general structure:

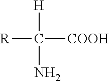

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “autologous”, as used herein, refers to something (e.g., a cell or cells) that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

A “bispecific antibody,” as used herein, refers to an antibody having binding specificities for at least two different antigenic epitopes. In some embodiments, the epitopes are from the same antigen. In some embodiments, the epitopes are from two different antigens. Methods for making bispecific antibodies are known in the art. For example, bispecific antibodies can be produced using recombinant technology using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Bolliger et al. (1993) Proc. Natl. Acad. Sci. U.S.A. 90:6444-48, Gruber et al. (1994) J. Immunol. 152:5368.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, |
| | polar residues | Gly |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, |
| | | Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci USA 87:2264-2268, modified as in Karlin & Altschul (1993) Applications and statistics for multiple high-scoring segments in molecular sequences. Proc Natl Acad Sci USA 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci USA 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI-BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to compositions or cells, refers to a single composition or cell of interest, or a population of compositions or cells of interest, at least partially isolated from other cell types or other cellular material with which it occurs in a culture or a tissue of origin.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm.

The default settings used for these programs are suitable for identifying substantially similar amino acid sequences for purposes of the presently disclosed subject matter. “Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Representative Embodiments

IIA. General Considerations

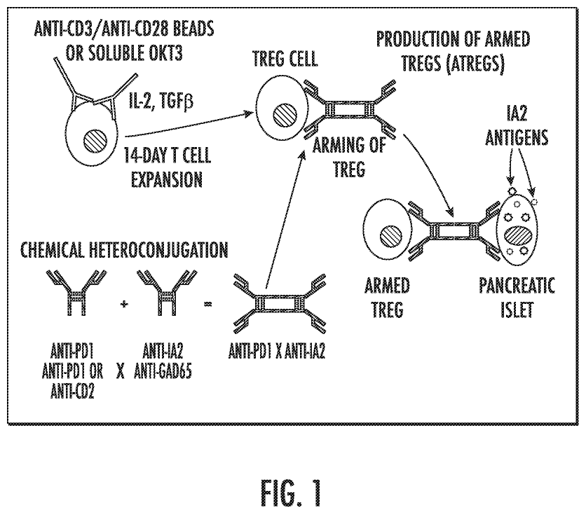

The ability of TREG cell therapy to induce tolerance in T1D patients can be enhanced if such TREG cells were targeted to the site of inflammation. To address this, bispecific antibody (BiAb) targeting of TREGs was used to improve delivery and efficacy of natural and induced polyclonal TREGs to the islet cell microenvironment (FIG. 1). Disclosed herein in some embodiments are novel methods to generate potent polyclonal TREGs, new assays to validate the function of the TREGs, and a BiAb armed TREG that will traffic specifically to the inflammatory site.

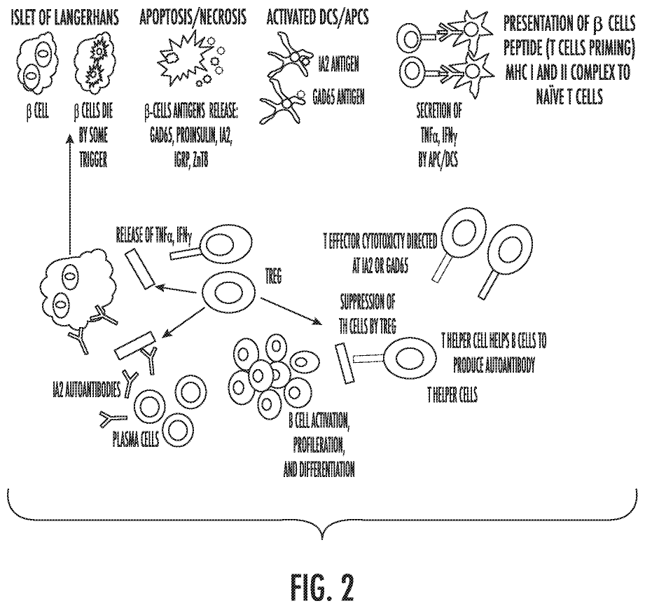

The development of immune destruction of pancreatic β islet cells responsible for producing insulin is unknown. The triggering events responsible for inducing immune responses to autoantigens IA2, GAD65, ZNT8, and insulin remains unclear. One defect that has emerged is that TREGs responsible for inhibiting autoimmune reactions have failed to prevent autoreactive T and B cells from damaging pancreatic islet cells. FIG. 2 shows the abnormal overactive helper T cells driving production of autoantibodies to IA2, GAD65, ZNT8, and insulin. The bars show where TREGs acts to inhibit autoreactive helper and cytotoxic T cells, and/or inhibit production of autoantibodies.

It is known that pre-T1D and T1D patients develop autoantibodies to the pancreatic β cell antigens IA2, GAD65, ZNT8, and [pro-insulin] that predict the development of T1D. Disclosed herein in some embodiments is a new unreported IA2 antigen-induced in vitro primary specific antibody (Ab) synthesis assay to measure specific anti-islet cell antigen IA2 Ab synthesis by normal donor peripheral blood mononuclear cells (PBMC). This assay assesses whether TREGs can suppress specific anti-IA2 Ab synthesis. Another assay in accordance with the presently disclosed subject matter is a transwell system that separates inflammatory Th₁ cells from the TREGs, thereby evaluating TREG suppression of Th₁ activated inflammatory cytokines in an antigen-independent manner. The presently disclosed subject matter also measures the ability of TREGs to suppress pokeweed mitogen (PW) or OKT3 activated in vitro polyclonal immunoglobulin (Ig) synthesis.

II.B. Representative Treatment Methods and Compositions

In some embodiments, the presently disclosed subject matter provides methods and compositions for treating a disease and/or disorder associated with inflammation. In some embodiments, the compositions are for use in preparing a medicament for treating a disease or disorder associated with inflammation.

In some embodiments, the method comprising administering to the subject an effective amount of a composition comprising a targeted activated regulatory T cell (AT_{REG}), which selectively binds a cell associated with disease and/or disorder associated with inflammation in the subject, to thereby treat the disease and/or disorder associated with inflammation in the subject. In some embodiments, the targeted activated regulatory T cell (AT_{REG}) is a bispecific antibody (BiAb) armed activated regulatory T cell (AT_{REG}). By “associated with inflammation” it is meant a disease or disorder where inflammation is present, for example, inflamed tissue is present. In some embodiments, the disease and/or disorder associated with inflammation comprises an autoimmune disease, optionally Type 1 diabetes, a graft-vs-host disease, an organ graft rejection. Thus, disclosed herein are methods for treating Autoimmune disease, Graft-vs-host disease, and/or Organ graft rejection (collectively AGO) and/or other diseases and/or disorders associated with inflammation.

In some embodiments, the disease or disorder is an infection, optionally a COVID19 infection. In some embodiments, the disease or disorder is inflammatory acute respiratory distress syndrome, such as might be associated with an infection, such as a COVID19 infection. A subject having any combination of the foregoing diseases can also be treated in accordance with the presently disclosed subject matter.

In some embodiments, the method comprises (a) isolating peripheral blood mononuclear cells from a subject suffering from a disease and/or disorder associated with inflammation; (b) arming a population of T regulatory cells (T_{REG}s) with a bispecific antibody (BiAb) directed at a TREG cell and to target an antigen of a cell associated with the disease and/or disorder associated with inflammation in the subject, under conditions wherein: (i) generation of T regulatory cells (T_{REG}s) occurs; (ii) bispecific antibody are used to arm T_{REG} cells (AT_{REG}s) and target the cell having the antigen, optionally an autoimmune antigen (such as IA2, GAD65, ZNT8, pro-insulin, or other autoantigen) or an infectious agent antigen (such as a COVID19 antigen); (iii) AT_{REG}s bind to the cell; and (iv) suppression of inflammatory activity by immune cells in the subject by AT_{REG} occurs; and (c) infusing a composition comprising the AT_{REG}s armed with a bispecific antibody into the subject, thereby treating the subject.

In some embodiments, the method comprises infusing AT_{REG}s intravenously and/or directly injecting AT_{REG}s into an affected organ and/or site with or without an additional therapeutic agent. In some embodiments, the additional therapeutic agent is IL-2, immune suppressive cytokines, immunosuppressive agents, and/or immunosuppressive monoclonal antibodies. In some embodiments, the method further comprises administering an effective amount of an additional therapeutic agent contemporaneously with or after the administering of the targeted activated T cell. In some embodiments, the additional therapeutic agent is an immunotherapy treatment. In some embodiments, the immunotherapy agent is selected from the group comprising an anti-CD52 antibody, an anti-CD20 antibody, an anti-CD20 antibody, anti-CD47 antibody an anti-GD2 antibody, a radiolabeled antibody, an antibody-drug conjugate, a cytokine, polysaccharide K and a neoantigen; optionally wherein said cytokine is an interferon, an interleukin, or tumor necrosis factor alpha (TNF-α), further optionally where said cytokine is selected from the group comprising IFN-α, INF-γ, IL-2, IL-12 and TNF-α. In some embodiments, the immunotherapy agent is selected from the group comprising Alemtuzumab, Ofatumumab, Rituximab, Zevalin, Adcetris, Kadcyla and Ontak. In some embodiments, the immunotherapy agent is selected from the group comprising a PD-1 inhibitor, a PD-L1 inhibitor, a CTLA-4 inhibitor, an IDO inhibitor, and a CCR7 inhibitor.

In some embodiments, the AT_{REG}s have been induced by ex vivo stimulation with an anti-CD3 Mab/IL-2 and/or anti-CD3/anti-CD28 in combination with rapamycin and/or temsirolimus with TGF-beta, optionally in a range of 1 ng/ml to 200 ng/ml, or any value within the range. In some embodiments, the T_{REG}s have been induced and/or maintained by an ex vivo treatment or by an in vivo treatment of the subject with a checkpoint inhibitor antibody selected from the group consisting of anti-PD1 (CD279), anti-PDL1 (CD274 or B6 B7-H1), anti-PDL2 (CD273), anti-CTLA4 (CD152), or any combination thereof, wherein the checkpoint inhibitor antibody or antibodies enhance suppressor activity in the T_{REG}s. In some embodiments, the potency and phenotype of T_{REG}s can be induced, enhanced, and/or maintained by in vitro arming or in vivo arming the TRegs with a BiAb with an anti-T cell partner being a checkpoint inhibitor agonistic to induce suppressor activity.

In some embodiments, the AT_{REG}s are from an autologous donor to the patient and/or are from an allogeneic donor to the patient. In some embodiments, the T_{REG} is a CD4⁺/FoxP3⁺ cell or a CD8⁺/FoxP3⁺ cell.

In some embodiments, arming doses provide 50% suppression at E:T of 1:1 to 5:1 in an immune suppression assay, including 2:1, 3:1, and 4:1. In some embodiments, the T_{REG}s can be armed with BiAbs doses ranging from 0.01 ng/million to 500 ng/million T_{REG}s, and/or any value within this range.

In some embodiments, the BiAb comprises two monoclonal antibodies. In some embodiments, the BiAb is directed at any non-activating T cell antigen. In some embodiments, the AT_{REG} is targeted at any surface antigen on pancreatic islet or organ cell being damaged by an inflammatory process, optionally IA2, GAD65, or ZNT8, or is targeted at a COVID 19 antigen, optionally a SAR-CoV2 antigen, further optionally spike, S1 receptor binding domain, nucleocapsid, or membrane antigen. In some embodiments, the construct is a trispecific antibody targeting a T cell antigen, a checkpoint mAb on the T cells, and IA2, GAD65, ZNT8, or other GAO antigen. In some embodiments, the BiAb is directed at any surface antigen on pancreatic islet or organ cell being damaged by an inflammatory process (e.g. overexpressed antigens AGO patients). In some embodiments, T cell suppression is needed in patients suffering from diseases associated with AGO. In some embodiments, the AT_{REG}s can be frozen and thawed for use in a subject in need of such therapy.

In some embodiments, binding of the AT_{REG}s induces secretion of one or more cytokines and/or chemokines by the AT_{REG}s that attenuate other cell populations in the inflammatory microenvironment to decrease inflammatory responses to autoantigens. In some embodiments, the presently disclosed methods comprise administering at least 1 infusion of AT_{REG}s to the patient. In some embodiments, the AT_{REG}s are co-administered to patients with AGO and/or other diseases and/or disorders associated with inflammation, optionally wherein the AT_{REG}s are co-administered with a second therapy designed to treat the AGO and/or other diseases and/or disorders associated with inflammation.

In some embodiments, the presently disclosed subject matter provides a pharmaceutical composition comprising, consisting essentially of, or consisting of an effective amount of a targeted activated T cell, such as a bispecific antibody armed activated regulatory T cell (AT_{REG}), which selectively binds a cell associated with disease and/or disorder associated with inflammation in the subject; and a pharmaceutically acceptable carrier. In some embodiments, the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof. In some embodiments, the composition is for use in treating a disease and/or disorder associated with inflammation in the subject, alone or in combination with an effective amount of an additional therapeutic agent.

In some embodiments, the presently disclosed subject matter provides for the use of an effective amount of a targeted activated T cell, such as a bispecific antibody armed targeted activated regulatory T cell (AT_{REG}), which selectively binds a cell associated with disease and/or disorder associated with inflammation in the subject, to treat the disease and/or disorder associated with inflammation in the subject or the preparation of a medicament for treating disease and/or disorder associated with inflammation in the subject. In some embodiments, the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof.

In some embodiments, the presently disclosed subject matter provides a composition for treating a mammal suffering from autoimmune diseases, graft-vs-host diseases, and/or organ graft rejection (collectively AGO) comprising AT_{REG}s targeting checkpoint antigens on the mammal's T cells and autoimmune antigen targets in inflamed tissue of the mammal.

In some embodiments, the subject is a mammalian subject. In some embodiments, the composition comprising the targeted activated T cell and/or the additional therapeutic agent is/are adapted for administration for the treatment of a subject by intravenous administration, intrathecal injection, peritoneal injection, or direct injection into a site of inflammation.

Any suitable or desired approach for producing activated T cells as would be apparent to one of ordinary skill in the art upon a review of the instant disclosure can be employed. Representative approaches are provided in the EXAMPLES. In some embodiments, the targeted activated T cells are produced from an apheresis product. In some embodiments, the targeted activated T cells are produced from an apheresis product by anti-CD3 stimulation (such as through the use of a soluble OKT3 dose of 20 ng/ml) in the presence of IL-2, optionally at a range of about 20 to about 200 IU/ml, In some embodiments, co-stimulated T cells are produced from an apheresis product by co-stimulation with anti-CD3/anti-CD28 coated beads in the presence or absence of IL-2 (5-200 IU/ml, optionally 20-200 IU/ml, including 5, 10, 15, 20, 25, 30, 35, 40, 45, 50, 55, 60, 65, 70, 75, 80, 85, 90, 95, 100, 105, 110, 115, 125, 130, 135, 140, 145, 150, 155, 160, 165, 170, 175, 180, 185, 190, 195, 2001 U/ml), such as at bead to cell ratios from 1:3 to 3:1. Alternatively, co-stimulated T cells or T cell subsets are co-stimulated with anti-CD3/anti-CD2/anti-CD28 coated beads in the presence or absence of IL-2, with IL-2 in the amounts mentioned immediately above when present. Representative techniques are also disclosed in U.S. Pat. No. 7,763,243, U.S. Patent Application Publication No. 2018/0282693, U.S. Patent Application Publication No. 2018/0243341, and U.S. Patent Application Publication No. 2019/0343954, each of which is hereby incorporated by reference in its entirety.

As used herein, the term “targeted activated T cell” refers to a T cell that has/have been made selective for a target antigen, such as by being stimulated with antigenic material, such as an autoantigen or an antigen from an infectious agent. The targeted activated T cell can be autologous or allogeneic to the subject. In accordance with the presently disclosed subject matter, an AT_{REG} is targeted at any surface antigen on pancreatic islet or organ cell being damaged by an inflammatory process, optionally IA2, GAD65, or ZNT8, or is targeted at a COVID 19 antigen, optionally a SAR-CoV2 antigen, further optionally spike, S1receptor binding domain, nucleocapsid, or membrane antigen.

Further, targeted activated T cells can be made selected for a target antigen by a T cell effector. An example of a T cell effector in accordance with the presently disclosed subject matter is a bispecific antibody. However, any suitable or desired T cell or T cell effector as would be apparent to one or ordinary skill in the art upon a review of the instant disclosure can be employed. In some embodiments, the T cell or T cell effector is selected from the group comprising peripheral blood mononuclear cells, unfractionated CD3+ T cells, CD4+ T cells, CD8+ T cells, and combinations thereof. In some embodiments, the T_{REG} is a CD4⁺/FoxP3⁺ cell or a CD8⁺/FoxP3⁺ cell.

Targeted activated T cells are prepared and maintained in any suitable medium as would be apparent to one of ordinary skill in the part upon a review of the instant disclosure. In some embodiments, the culture comprises a basal medium. Other representative media and media ingredients are described in the EXAMPLES and/or would be apparent to one of ordinary skill in the art upon a review of the instant disclosure, including but not limited to known and/or commercially available media. By way of example and not limitation, other media and media components include but are not limited to RPMI 1640, Ex vivo 15, Ex Vivo 20, Aim V, CTS OpTmizer T-Cell Expansion SFM, LymphoONE, and/or other T cell culture or equivalent and other complete media in the presence or absence of serum, such as about 2 to about 10% fetal calf serum or human serum, including about 2%, about 3%, about 4%, about 5%, about 6%, about 7%, about 8%, about 9%, and about 10% serum, or artificial serum components (serum free media). In some embodiments, the culture comprises a media comprising RPMI1640 supplemented with 2% human serum. Commercial sources for media include Thermo Fisher Scientific (Hampton, N.H.), MilliporeSigma (Burlington, Mass.) and Sigma-Aldrich (St. Louis, Mo.). Ex vivo 15 and Ex vivo 20 are commercially available under the trademarks X-VIVO™15 and X-VIVO™20 (Lonza Walkersville, Inc., Walkersville, Md.); Aim V is commercially available under the trademark AIM V™ (Life Technologies Corporation, Carlsbad, Calif.), CTS OpTmizer T-Cell Expansion SFM is commercially available under the trademark CTS™ OPTMIZER™ T Cell Expansion SFM (Life Technologies Corporation, Carlsbad, Calif.), and LymphoOne is commercially available under the trademark LYMPHOONE™ (Takara Bio Inc., Kusatsu, Japan).

Any suitable or desired bispecific antibody as would be apparent to one of ordinary skill in the art upon a review of the instant disclosure can be employed. In some embodiments, the bispecific antibody used to arm the targeted activated T cell is selected from the group including but not limited to a chemically heteroconjugated bispecific antibody or recombinant bispecific antibodies of any configuration (e.g., univalent, bivalent, or multi-valent bispecific antibodies directed at T cells and at the target antigen). In some embodiments, the bispecific antibody can be directed to any surface antigen on pancreatic islet or organ cell being damaged by an inflammatory process, optionally IA2, GAD65, or ZNT8, or is targeted at a COVID 19 antigen, optionally a SAR-CoV2 antigen, further optionally spike, S1 receptor binding domain, nucleocapsid, or membrane antigen. In some embodiments, the AT_{REG}s have been induced by ex vivo stimulation with an anti-CD3 Mab/IL-2 and/or anti-CD3/anti-CD28 in combination with rapamycin and/or temsirolimus with TGF-beta, optionally in a range of 1 ng/ml to 200 ng/ml. In some embodiments, the T_{REG}s have been induced and/or maintained by an ex vivo treatment or by an in vivo treatment of the subject with a checkpoint inhibitor antibody selected from the group consisting of anti-PD1 (CD279), anti-PDL1 (CD274 or B6 B7-H1), anti-PDL2 (CD273), anti-CTLA4 (CD152), or any combination thereof, wherein the checkpoint inhibitor antibody or antibodies enhance suppressor activity in the T_{REG}s. In some embodiments, the potency and phenotype of T_{REG}s can be induced, enhanced, and/or maintained by in vitro arming or in vivo arming the TRegs with a BiAb with an anti-T cell partner being a checkpoint inhibitor agonistic to induce suppressor activity.

Techniques and examples of bispecific antibodies are disclosed in the art. See, for example, U.S. Pat. No. 7,763,243, U.S. Patent Application Publication No. 2018/0282693, U.S. Patent Application Publication No. 2018/0243341, and U.S. Patent Application Publication No. 2019/0343954, each of which is hereby incorporated by reference in its entirety. Additional examples are also described, such as in Thakur et al., Oncoimmunology. 2018 Aug. 27; 7(12); Vaishampayan et al., Prostate Cancer. 2015; 2015:285193, Epub 2015 Feb. 23; Lum et al., Clin Cancer Res. 2015 May 15; 21(10):2305-14, Epub 2015 Feb. 16; Zitron et al., BMC Cancer. 2013 Feb. 22; 13:83; Yankelevich et al., Pediatr Blood Cancer. 2012 Dec. 15; 59(7):1198-205; Zhao et al., J Appl Physiol 104:1793-1800, 2008; Sen et al., J Hematother Stem Cell Res 2001 10:247-60; Lum et al., Clinical Breast Cancer, Vol. 4, No. 3, 212-217, 2003; Lum, Expert Opin. Drug Discov. (2008) 3(9):1-17; Lum et al., Biol Blood Marrow Transplant 18: 1012-1022 (2012); Lum et al., Biol Blood Marrow Transplant 19 (2013) 925-933. In some embodiments, the BiAb used to arm the targeted activated T cell is a chemically heteroconjugated bispecific antibody or a recombinant bispecific antibody of any configuration.

The presently disclosed subject matter is also directed to methods of administering the compositions of the presently disclosed subject matter to a subject.

Pharmaceutical compositions in accordance with the presently disclosed subject matter are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

The presently disclosed subject matter encompasses the preparation and use of pharmaceutical compositions for treatment of the diseases and disorders disclosed herein. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient. It can generally be stated that a pharmaceutical composition comprising the T cells described herein may be administered at a dosage of 10⁴ to 10¹ cells/kg body weight, optionally 10⁵ to 10⁶ cells/kg body weight, including all integer values within those ranges. T cell compositions may also be administered multiple times at these dosages. The cells can be administered, for example, by using infusion techniques that are commonly known in immunotherapy (see, e.g., Rosenberg et al., New Eng. J. of Med. 319:1676, 1988). The optimal dosage and treatment regime for a particular subject can readily be determined by one skilled in the art of medicine by monitoring the subject for signs of disease and adjusting the treatment accordingly.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pa., United States of America and/or Gennaro (ed.) (2003) Remington: The Science and Practice of Pharmacy, 20th edition Lippincott, Williams & Wilkins, Philadelphia, Pa., United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the compositions of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, N.Y., United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, N.Y., United States of America; and Ausubel et al. (1995) Current Protocols in Molecular Biology, Greene Publishing.

III.C. Antibody Formats and Preparation Thereof

Any suitable bispecific antibody and technique for the production thereof as would be apparent to one of ordinary skill in the art upon a review of the instant disclosure falls within the scope of the presently disclosed subject matter. In some embodiments, the presently disclosed subject matter employs bispecific antibodies (BiAbs) produced by chemical joining of two monoclonal antibodies. Examples of bispecific antibodies and techniques for producing bispecific antibodies are known the art and have been described in several reviews, along with their respective target antigens and T cell antigens. Representative reviews include Thakur, A., and Lum, L. G.: Cancer therapy with bispecific antibodies: Clinical experience. Current Opinion and Molecular Therapeutics 12:340-349, 2010; Lum, L. G., and Thakur, A.: Bispecific Antibodies for Arming Activated T Cells and Other Effector Cells for Tumor Therapy. Book Chapter in: Bispecific Antibodies. Kontermann, R. E. (ed). Germany: Springer Heidelberg, 2011, pp. 243-271; Lum, L. G., and Thakur, A.: Targeting T Cells with Bispecific Antibodies for Cancer Therapy: A Review. BioDrugs 25:365-379, 2011; and Thakur, A., Huang, M., Lum, L. G.: Bispecific antibody based therapeutics: Strengths and challenges. Blood Reviews, 2018 (Impact 6.6).

Representative BiAbs include but are not limited to whole IgG-based BiAbs, trifunctional BiAbs, BiAb Format based on single-chain variable fragment. Representative U.S. patents relating to BiAbs and production thereof include U.S. Pat. Nos. 10,550,193; 10,519,247; 10,294,300; 10,239,951; and 10,179,819, each of which is herein incorporated by reference in its entirety.

In some embodiments, the presently disclosed subject matter relates to targeting PD1 and to the use of anti-PD1 antibodies. Human PD1 is GENBANK® Accession No. NP_005009.2, encoded by GENBANK® Accession No. NM_005018.2. The following documents, including patent documents, relate to PD1 and to antibodies directed to PD1 and the disclosure of each of these documents is herein incorporated by reference. U.S. Patent Application Publication No. 2017/0247454. Exemplary anti-PD1 antibodies include pembrolizumab (also called lambrolizumab and MK-3475, MERCK; covered by U.S. Pat. No. 8,952,136), nivolumab (BMS-936558, BRISTOL-MYERS SQUIBB; EP 2 161 336 and U.S. Pat. No. 8,008,449), AMP-224 (MERCK), and pidilizumab (CT-011, CURETECH LTD.). Anti-PD1 antibodies are commercially available, for example from ABCAM® (AB137132), BIOLEGEND® (EH12.2H7, RMP1-14) and AFFYMETRIX EBIOSCIENCE (J105, J116, MIH4). U.S. Patent Application Publication No. 2010/0028330 (see e.g., Table 1-3); U.S. Pat. No. 9,217,034; U.S. Patent Application Publication No. 2019/0321466; U.S. Pat. No. 10,513,558; U.S. Patent Application Publication No. 2019/0382491; WO 2016/062722. Other Anti-PD-1 antibodies are known in the art. Exemplary anti-PD-1 antibodies include: nivolumab, pembrolizumab, pidilizumab and MPDL3280A.

In some embodiments, the presently disclosed subject matter relates to targeting PD-L1 and to the use of anti-PD-L1 antibodies. Human PD-L1 is GENBANK® Accession No. NP_054862.1, encoded by GENBANK® Accession No. NM_014143.4 (isoform a precursor) and GENBANK® Accession No. NP_001254635.1 encoded by GENBANK® Accession No. NM_001267706.1. The following documents, including patent documents, relate to PD1 and to antibodies directed to PD1 and the disclosure of each of these documents is herein incorporated by reference. U.S. Patent Application Publication No. 2017/0247454. Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1). PCT International Publication No. WO 2016/062722 (“Anti-PD-L1 and anti-PD1 therapeutic blocking antibodies are being trialed and have shown clinical benefit in a number of tumor types including lung cancer, melanoma, renal cell carcinoma, bladder cancer, gastric cancer, head and neck cancer, etc.; but only a minority of patients respond to these therapies (e.g. see, Brahmer et al, New Engl. J. Med. 366(26):2455-2465, 2012; Harvey. Clinical Pharmacology & Therapeutics 96(2): 214-223, 2014”). Anti-PD-L1 antibodies are known in the art. Exemplary anti-PD-L1 antibodies include: MEDI4736 (durvalumab), MPDL3280A, BMS936559, 2.7A4, AMP-714 and MDX-1105. PCT International Patent Application No. 2020/0190193 (PD-1 SINGLE DOMAIN ANTIBODIES AND THERAPEUTIC COMPOSITIONS THEREOF).

In some embodiments, the presently disclosed subject matter relates to targeting CTLA4 and to the use of anti-CTLA4 antibodies. Human CTLA4 is GENBANK® Accession No. NP_005205.2, encoded by GENBANK® Accession No. NM_005214.5. The following documents, including patent documents, relate to CTLA4 and to antibodies directed to CTLA4 and the disclosure of each of these documents is herein incorporated by reference. U.S. Patent Application Publication No. 2017/0247454 and PCT International Publication No. WO 2016/062722. Also, exemplary anti-CTLA4 antibodies include ipilimumab (Bristol-Myers Squibb) and tremelimumab (PFIZER). Anti-PD1 antibodies are commercially available, for example from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89). Exemplary anti-CTLA-4 antibodies include: tremelimumab and ipilimumab, also termed MDX-010 (or BMS-734016).

In some embodiments, the presently disclosed subject matter relates to targeting CD2 and to the use of anti-CD2 antibodies. Human CD2 is GENBANK® Accession No. NP_001315538.1, encoded by GENBANK® Accession No. NM_001328609.2. The following documents, including patent documents, relate to CD2 and to antibodies directed to CD2 and the disclosure of each of these documents is herein incorporated by reference. U.S. Pat. No. 5,798,229 (Bispecific molecules recognizing lymphocyte antigen CD2 and tumor antigens); U.S. Pat. No. 7,250,167 (Method of treatment with anti-CD2 antibody LO-CD2b). Exemplary anti-CD2 antibodies include: Santa Cruz Biotechnology Catalogue No. sc-19638; Abcam Catalogue Nos: ab131276, an227698, ab119993, ab193344 and others; Sigma-Aldrich clone RPA-2.10; Amevive CD2 IgG1 fusion Immunomodulatory (alefacept).

In some embodiments, the presently disclosed subject matter relates to targeting PDL2 and to the use of anti-PDL2 antibodies. Human PDL2 is GENBANK® Accession No. NP_079515.2, encoded by GENBANK® Accession No. NM_025239.4. A representative commercially available PDL2 antibody is available under Sigma-Aldrich Catalogue No. SAB3500395. The following documents, including patent documents, relate to PDL2 and to antibodies directed to PDL2 and the disclosure of each of these documents is herein incorporated by reference. U.S. Patent Application Publication Nos. 2017/0274073, 2017/0290913, 2017/0349666, and 2018/0303936; U.S. Pat. No. 10,556,957. Anti-PDL2 antibodies are commercially available, e.g., Abcam PLC (including but not limited to catalog numbers ab256386, ab187662, ab234187, ab110182, ab231145, and ab21107), Lifespan Biosciences (including but not limited to catalog numbers LS-B16021, LS-A10667, LS-B14994, and LS-B16020), Santa Cruz Biotechnology (including but not limited to catalog number sc-80285) Sigma-Aldrich (including but not limited to catalog numbers SAB3500395, SAB1411458, and SAB4700812).

In some embodiments, the presently disclosed subject matter relates to targeting IA2 and to the use of anti-IA2 antibodies. Human IA2 (receptor-type tyrosine-protein phosphatase-like N isoform 1 precursor) is GENBANK® Accession No. NP_002837.1, encoded by GENBANK® Accession No. NM_002846.4. The following documents, including patent documents, relate to IA2 and to antibodies directed to IA2 and the disclosure of each of these documents is herein incorporated by reference. PCT International Publication No. WO 2000/012558 A1, with disclosure of human monoclonal antibodies directed against the islet cell antigen ia-2 and reference to a deposit DSM ACC2365; U.S. Patent Application Publication No. 2014/0030234. Also, anti-IA2 antibodies are commercially available, e.g., Abcam PLC (including but not limited to catalog numbers ab207750, ab251479, ab198279, and ab244039), Lifespan Biosciences (including but not limited to catalog numbers LS-B5395, LS-B13925, LS-C62171, and LS-C324421), Santa Cruz Biotechnology (including but not limited to catalog numbers sc-390101, sc-130570, and sc-135672) Sigma-Aldrich (including but not limited to catalog number SAB2500525).

In some embodiments, the presently disclosed subject matter relates to targeting SARS-Cov-2 antigens (e.g., spike, S1 receptor binding domain, nucleocapsid, and membrane antigens) and to the use of anti-SARS-Cov-2 antibodies. The following documents, including patent documents, relate to SARS-Cov-2 antigens and to antibodies directed to SARS-Cov-2 antigens and the disclosure of each of these documents is herein incorporated by reference. GENBANK® Accession No. NC_045512.2 corresponds to the complete genomic nucleotide sequence of the severe acute respiratory syndrome coronavirus 2 isolate Wuhan-Hu-1. The amino acid sequence encoded by nucleotides 21563-25384 of GENBANK® Accession No. NC_045512.2 corresponds to the complete spike glycoprotein (S) polypeptide sequence of the severe acute respiratory syndrome coronavirus 2 isolate Wuhan-Hu-1. The amino acid sequence set forth in GENBANK® Accession No. QHD43416.1 is a full length S amino acid sequence of SARS-CoV-2 (also set forth in GENBANK® Accession No YP_009724390.1). It is encoded by nucleotides 21563-25384 of GENBANK® Accession No. NC_045512.2. Various subsequences of the S polypeptide can be employed for targeting with anti-SARS-CoV-2 antibodies (see e.g., Zheng & Son (2020) Cellular & Molecular Immunology 17:536-538. Other antigenic proteins encoded by the SARS-CoV-2 genome include the nucleocapsid (N) protein (GENBANK® Accession No. YP_009724397.2, encoded by nucleotides 28274-29533 of NC_045512.2). Anti-SARS-CoV-2 antibodies are described, for example, in Chinese Patent Application Publications CN 111153991 A and CN 11218458 A, each of which is incorporated herein in its entirety. Anti-SARS-CoV-2 antibodies are also commercially available from Sino Biological (including but not limited to catalog numbers 40591-MM43, 40592-MM57, 40592-R001, and 40590-D001 for anti-S polypeptide antibodies, and 40143-R004, 40143-MM05, and 40143-MM08 for anti-N polypeptide antibodies), Lifespan Biosciences (including but not limited to catalog numbers LS-C829120, LS-C-829810, LS-C829811, LS-C829813, LS-C829816, and LS-C829820), Santa Cruz Biotechnology (including but not limited to catalog number sc-65653), and Sigma-Aldrich (including but not limited to catalog numbers ZMS1075, ZMS1076, and ZHU1076). See also Grifoni et al. (2020) Cell Host & Microbe 27:671-680.

In some embodiments, the presently disclosed subject matter relates to targeting GAD65 gene products and to the use of anti-GAD65 antibodies. The following documents, including patent documents, relate to GAD65 antigens and to antibodies directed to GAD65 antigens and the disclosure of each of these documents is herein incorporated by reference. GENBANK® Accession Nos. NP_000809.1 and NP_001127838.1 provide amino acid sequences of exemplary human GAD65 gene products. These exemplary amino acid sequences are encoded by the exemplary nucleic acid sequences disclosed as GENBANK® Accession Nos. NM_000818.2 and NM_001134366.2, respectively. Anti-GAD65 antibodies are described, for example, in U.S. Patent Application Publication Nos. 2005/0044588, 2016/0032016 and U.S. Pat. Nos. 8,211,430 and 8,816,047, each of which is incorporated herein in its entirety. See also Verge et al. (1998) Diabetes 47(12):1857-1866. Anti-GAD65 antibodies are also commercially available from Abcam PLC (including but not limited to catalog numbers ab239372, ab183999, ab270037, and ab26113), Lifespan Biosciences (including but not limited to catalog numbers LS-B3937, LS-B9984, LS-A12397, and LS-C185356), Santa Cruz Biotechnology (including but not limited to catalog numbers sc-377145, sc-32270, sc-130569, and sc-354180), and Sigma-Aldrich (including but not limited to catalog number SAB4300643).

In some embodiments, the presently disclosed subject matter relates to targeting ZNT-8 gene products and to the use of anti-ZNT-8 antibodies. The following documents, including patent documents, relate to ZNT-8 antigens and to antibodies directed to ZNT-8 antigens and the disclosure of each of these documents is herein incorporated by reference. GENBANK® Accession Nos. NP_001166282.1 NP_001166284.1 NP_001166285.1 NP_001166286.1 and NP_776250.2 provide amino acid sequences of exemplary human ZNT-8 gene products. These exemplary amino acid sequences are encoded by the exemplary nucleic acid sequences disclosed as GENBANK® Accession Nos. NM_001172811.2, NM_001172813.2, NM_001172814.2, NM_001172815.2, and NM_173851.3, respectively. Anti-ZNT-8 antibodies are described, for example, in U.S. Pat. No. 9,023,984, which is incorporated herein in its entirety. Anti-ZNT-8 antibodies are also commercially available from Abcam PLC (including but not limited to catalog numbers ab254577, ab244550, ab136990, and ab229954), Lifespan Biosciences (including but not limited to catalog numbers LS-C296475, LS-C111163, LS-C296473, and LS-C501317), Santa Cruz Biotechnology (including but not limited to catalog number sc-514715), and Sigma-Aldrich (including but not limited to catalog number SAB2105710).

In some embodiments, the presently disclosed subject matter relates to targeting pro-insulin polypeptides and to the use of anti-pro-insulin antibodies. The following documents, including patent documents, relate to pro-insulin antigens and to antibodies directed to pro-insulin antigens and the disclosure of each of these documents is herein incorporated by reference. GENBANK® Accession Nos. NP_000198.1, NP_001172026.1, NP_001172027.1, and NP_001278826.1, provide amino acid sequences of exemplary human pro-insulin polypeptides. These exemplary amino acid sequences are encoded by the exemplary nucleic acid sequences disclosed as GENBANK® Accession Nos. NM_000207.3, NM_001185097.21, NM_001185098.2, and NM_001291897.2, respectively. Anti-pro-insulin antibodies are described, for example, in U.S. Patent Application Publication Nos. 2005/0164303 and 2006/0117412, each of which is incorporated herein in its entirety. Anti-pro-insulin antibodies are also commercially available from Abcam PLC (including but not limited to catalog numbers ab243141, ab8304, ab8305, and ab255745), Lifespan Biosciences (including but not limited to catalog numbers LS-C828676, LS-C-828677, and LS-C829060), Santa Cruz Biotechnology (including but not limited to catalog numbers sc-8033, sc-52035, sc-52037, and sc-9167), and Sigma-Aldrich (including but not limited to catalog number WH0003630M1). In some embodiments, one or more antibodies or fragments thereof are used.

In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab. Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments. In some embodiments, the specific binding molecule is a single-chain variable (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies. Fragments within the scope of the term “antibody” include those produced by digestion with various proteases, those produced by chemical cleavage and/or chemical dissociation and those produced recombinantly, so long as the fragment remains capable of specific binding to a target molecule (i.e., comprise at least one paratope).

Other representative patent documents disclosing techniques relating to antibody production include the following, all of which are herein incorporated by reference in their entireties: PCT International Patent Application Publication Nos. WO 1992/02190 and WO 1993/16185; U.S. Patent Application Publication Nos. 2004/0253645, 2003/0153043, 2006/0073137, 2002/0034765, and 2003/0022244; and U.S. Pat. Nos. 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,643,759; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; 6,750,325; and 6,797,492.

### IV. Examples

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

A non-limiting example of AGO and/or other diseases and/or disorders associated with inflammation is Type 1 diabetes (T1D). The ability of TReg cell therapy to induce tolerance in Type 1 diabetes (T1D) patients may be enhanced if such TReg cells were targeted to the site of inflammation. To address this, the presently disclosed subject matter relates to adapt bispecific antibody (BiAb) targeting of TRegs to improve delivery of natural and induced polyclonal TRegs to sites of inflammation including, but not limited to, the islet cell microenvironment. As such, the presently disclosed subject matter relates in some embodiments to novel methods to generate potent polyclonal TRegs, new assays to validate the function of the TRegs, and BiAb-armed TRegs that traffic to inflammatory sites.

The presently disclosed subject matter provides an IA2 antigen-induced in vitro primary specific antibody (Ab) synthesis assay to measure specific anti-islet cell antigen IA2 antibody synthesis by normal donor peripheral blood mononuclear cells (PBMC). This assay, which uses IA2 antigen to stimulate in vitro primary specific anti-IA2 IgM and IgG Ab synthesis as measured by B cell EliSpots, assesses whether regulatory T cells (T_{REG}s) can suppress specific anti-IA2 Ab synthesis. In pre-clinical studies, methods have been developed to generate polyclonal natural and induced T_{REG}s cells. Several antigen-specific and non-specific assays were used, re-purposed, or newly developed to validate the function of T_{REG}s. One new assay is a transwell system that separates inflammatory Th1 cells from the T_{REG}s, thereby evaluating T_{REG} suppression of Th1 activated inflammatory cytokines in an antigen-independent manner. The ability of T_{REG}s to suppress pokeweed mitogen (PWM) activated in vitro polyclonal immunoglobulin (Ig) synthesis is also examined.

While not wishing to be bound by any particular theory of operation, inducible T_{REG} are functionally potent, are expanded to clinically applicable doses, are clinically safe, and suppress specific and polyclonal antibody synthesis.

### EXAMPLES 1 TO 3

EXAMPLES 1 to 3 relate to the following aspects:

1. Confirming that the methods for producing improved natural and induced T_{REG}s from the PBMC of normal donors can be applied to PBMC from pre-T1D and T1D patients;

2. Developing bispecific antibodies (BiAbs) that target T_{REG}s (AT_{REG}s) to beta islet cells by:

(a) Non-activating anti-T cell antibodies as partners to anti-IA2 or anti-GAD65 to test for targeting without losing suppressor function or inducing cytotoxic function directed at pancreatic islet cells.

(b) Agonistic anti-T cell checkpoint anti-PD1 and anti-PDL1 antibodies as partners to anti-IA2 or anti-GAD65 that induce and/or maintain T_{REG} suppressor function without inducing cytotoxicity directed at pancreatic islet cells.

3. Testing the trafficking of AT_{REG}s in a xenogeneic pancreatic islet graft in the renal capsule of an immune-deficient mouse, and test AT_{REG} ability to block rejection of a pancreatic islet graft in an immune-competent mouse.

Targeted T_{REG}s are administered to patients with autoimmune disorders including but not limited to T1D patients. In some embodiments, multiple infusions of autologous T_{REG}s for the treatment of T1D patients and patients with other autoimmune disorders are employed. Achieving clinically effective tolerance in T1D patients, delaying the onset of T1D by initiating treatments in high risk T1DGC patients early after diagnosis, correcting the immune imbalance, and expanding adequate numbers of functionally effective T_{REG}s changes the paradigm in the treatment of patients with T1D, leading to the slowing or arrest of β cell destruction.

### Example 1

In this EXAMPLE, the first step in the approach is to functionally characterize and select candidate natural and induced T_{REG} cell substrates produced from methods currently being implemented, including various forms of activation (anti-CD3/IL-2, anti-CD3/anti-CD3 coactivation), various inhibitors (rapamycin, temsirolimus), various cytokine cocktails (IL-2, IL-4, TGF-β), and the presence or absence of PD1 receptor signaling. After selecting a suitable method for generating natural and inducible T_{REG}s, an ability to manufacture these two types of T_{REG}s using PBMC from normal controls, pre-T1D individuals, and T1D patients is compared. Manufacturing methods for clinical development are selected based on: a) T_{REG} phenotyping by flow cytometry for transcription factors (including FoxP3, Tbet) and effector molecules (including CD39, CD73); and b) functional testing for suppression in conventional mixed lymphocyte culture, and also in the novel assays disclosed herein, including: in vitro polyclonal Ig synthesis stimulated by pokeweed mitogen; in vitro anti-IA2 or anti-GAD65 specific Ab synthesis in combination with absence of cytotoxicity directed at islet cells; an EBV-driven assay11 of anti-IA2 IgG synthesis; and the transwell assay assessment of Th1 and T_{REG} cell interactions.

### Example 2

Chemically heteroconjugated BiAbs are produced to target T_{REG}s to beta islet cells expressing IA2, and GAD65 that target T_{REG}s to pancreatic islets by: a) using the suppression assays to screen for non-activating anti-T cell antibodies as partners to anti-IA2 or anti-GAD65 to test for targeting without losing suppressor function or inducing cytotoxic function at pancreatic islet cells/tissue; and b) using the suppression assays to screen and identify agonistic anti-T cell checkpoint anti-PD1 and anti-PDL1 antibodies as partners to anti-IA2 or anti-GAD65 that would target and bind as well as induce and/or maintain T_{REG} suppressor function without inducing cytotoxicity directed at pancreatic islet cells.

### Example 3

Test unarmed and armed T_{REG}s in two in vivo models: 1) In order to assess T_{REG} modulation of xenogeneic human-into-mouse GVHD, immune-deficient mice are infused with human Th1 cells to induce xenogeneic GVHD; candidate T_{REG}s are infused after establishment of Th1 cell engraftment to assess T_{REG} modulation of an ongoing inflammatory response; 2) Short-term pancreatic islet transplants in immune-competent mice to assess whether injection of AT_{REG}s can traffic to human antigen-bearing tissue.

Environment: A new 7,500 ft² cGMP facility was completed and validated for the manufacture of bispecific antibody armed activated T cells (BATs), CAR-T, and isolation of pancreatic islets. Located on the 2nd floor of Pinn Hall at UVA, Charlottesville, Va., United States of America, the facility contains six ISO 7 rooms and several support rooms, supply rooms, and offices. The PI has 3,500 ft² of research space on the 7th floor of West Complex at UVA housing a Bioplex system (Luminex), Cellular Technology L.T.D. ImmunoSpot® reader, 13 color Novacyte® Flow Cytometer (ACEA Biosciences Inc.), xCELLigence® RTCA Systems (ACEA Biosciences Inc.), WES for western blotting multiple proteins, and Milo for single cell westerns. Bioreactors for clinical expansion and incubators are already validated in the cGMP facility. Flow, genomic, and proteomic cores are readily available and are utilized as needed.

The presently disclosed subject matter relates in some embodiments to armed T_{REG}s that target antigens expressed on pancreatic islet cells.

Based on the data obtained from these studies, an IND for AT_{REG} infusions for patients with T1D is submitted. The phase I trial includes apheresis followed by infusions of the most promising candidate T_{REG} cell products. The phase I component of the trial involves a 3+3 dose escalation with multiple infusions of AT_{REG} given at doses ranging from 1-2×10⁶/kg to 500×10⁶/kg. The results drive the phase II component of the treatment strategy.

### EXAMPLES 4-9

Hypothesis: A hypothesis tested in the following Examples is that inducible T_{REG}s can be armed with bispecific antibodies (BiAb) directed at the T_{REG} cell on one end and directed at the injured or inflamed T1D pancreatic islet cells expressing IA2 or GAD65 on the other end so that the armed T_{REG} will traffic and bind to the inflamed or damaged pancreatic islets, leading to suppression of cellular and humoral immune responses to IA2 and GAD65 (FIG. 1).

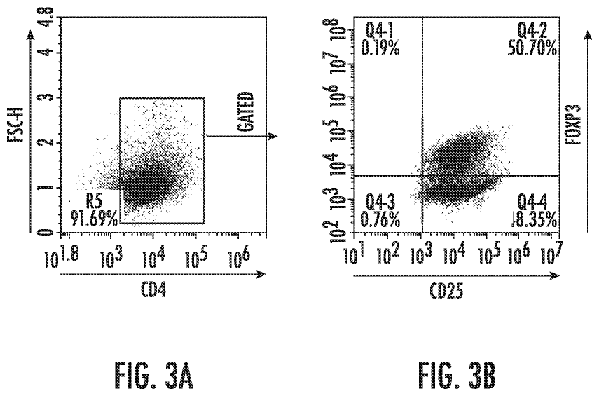

T_{REG} Growth: T_{REG}s were grown using soluble OKT3 (20 ng/ml), TGF-0 (100 ng/ml) in the presence rapamycin, and 100 IU IL-2, phenotype of T_{REG}s were confirmed by staining for CD4/CD25/FoxP3 (FIGS. 3A and 3B).

### Example 4

### T_{REG} Suppression of PW or OKT3 Stimulated Ig Synthesis

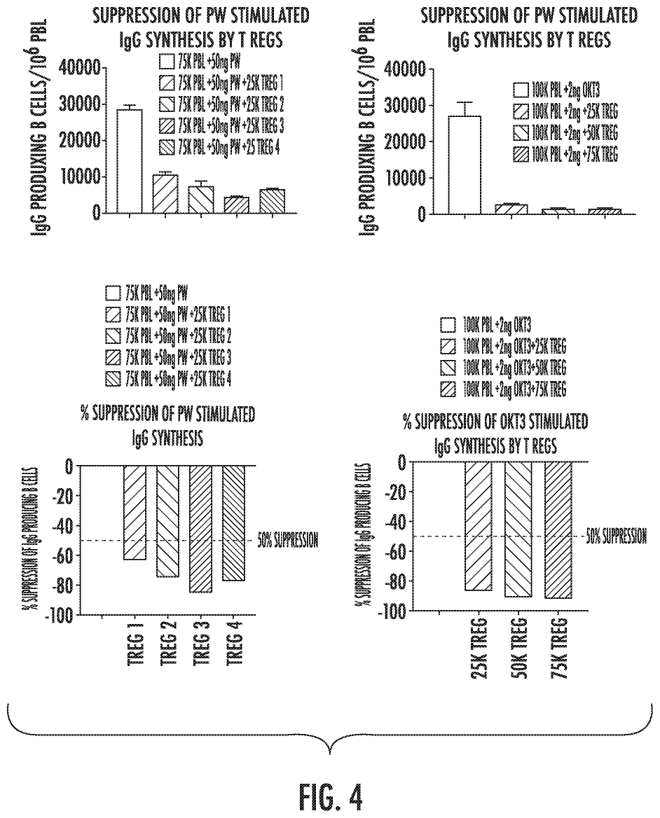

The addition of T_{REG}s from different individuals to autologous PBMC stimulated with PW suppressed the control (29,000 B cell EliSpots/10⁶ PBL or PBMC) from 60-88% with the addition of 0.025×10⁶ and 0.05×10⁶ to 10×10⁶ PBMC, respectively (FIG. 4). Similarly, T_{REG}s suppressed unrelated PBMC stimulated with OKT3 (20 ng/ml). The addition of 0.025 to 0.050×10⁶ T_{REG}s to 0.10×10⁶ PBMC suppressed Ig synthesis ranging from 85-95%.

### Example 5

### T_{REG} Suppression of IA2 Induced Specific Ab Synthesis

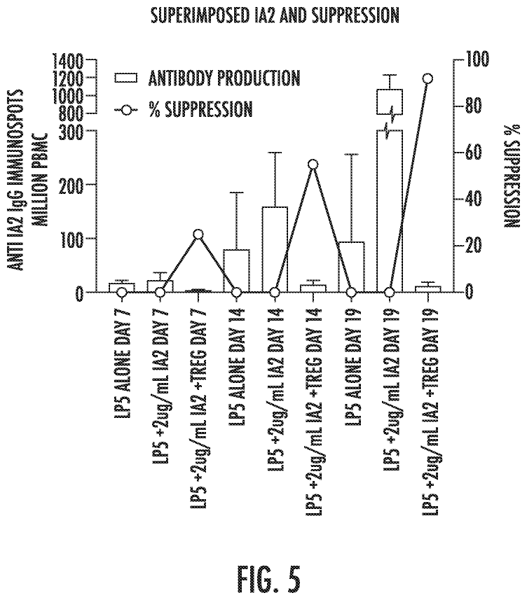

We developed an in vitro anti-IA2 Ab synthesis assay which measures single B cells secreting anti-IA2 Abs after IA stimulation. The optimal dose of IA2 to induce primary anti-IA2 Ab synthesis is 2 pg/ml based on a dose titration that was done to determine the kinetics of IgG and IgM anti-IA2 Ab (peak of IgM anti-IA2 synthesis ˜day 14. The optimal time for IgG anti-IA2 synthesis is day 19 (FIG. 5). 0.5×10⁶ T_{REG}s co-cultured with 2.0×10⁶ PBMC in the presence of 2 pg/ml of IA2 antigen for 19 days of culture suppressed anti-IA2 IgG synthesis >90%. Optimal IgM anti-IA2 synthesis at 14 days of culture was suppressed by 98%. The suppression mediated by T_{REG}s produced using various cytokine/drug combinations consistently suppressed anti-IA2 Ab synthesis.

### Example 6

### T_{REG} Suppression of Th₁ Cell Cytokine Release

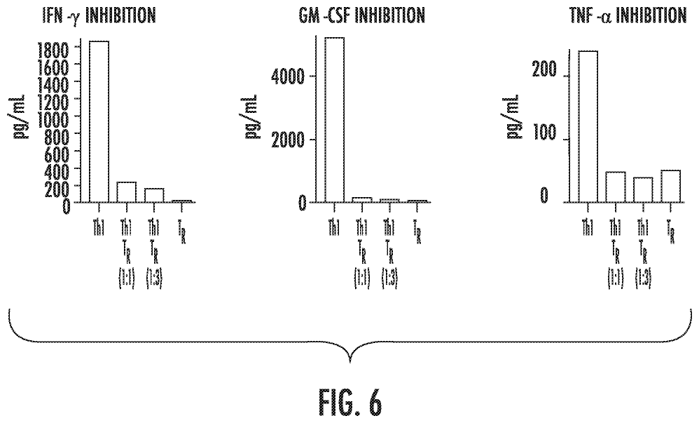

T-Rapa cells (Th₁) are manufactured to secrete Th₁ cytokines. The ability of T_{REG}s to suppress an array of Th₁ cytokines released on anti-CD3/anti-CD28 restimulation was assessed by adding T_{REG}s in a transwell assay that evaluates the production of T cell derived factors that would act on the Th₁ cells to suppress their ability to secrete Th₁ cytokines upon anti-CD3/anti-CD28 secretion. T_{REG}s added to T-Rapa cells consistently suppressed the secretion of Th₁ cytokines upon anti-CD3/anti-CD28 restimulation by 90% (FIG. 6).

### Example 7

### T_{REG} Suppression of Tetanus Toxoid (TT) Induced Specific Ab Synthesis

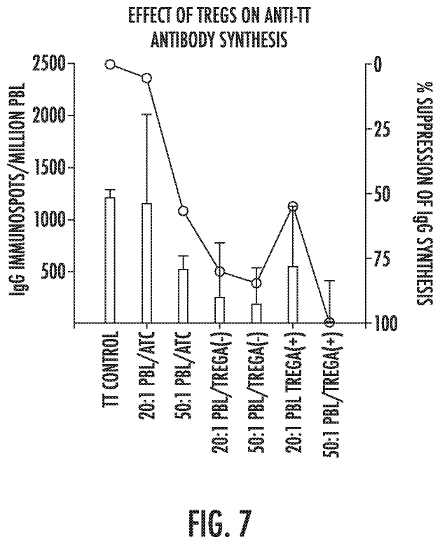

The addition of T_{REG}s to TT-induced specific Ab synthesis suppressed TT-specific B cell EliSpots. The left axis of FIG. 7 shows 1200 IgG anti-TT B cell EliSpots/10⁶ PBMC cultured. Activated T cells (ATC) added to the cultures suppressed at a PBMC/T_{REG} ratio of 50:1 by ˜₆₀%. T_{REG}s that were produced in the standard manner (T_{REG}s A−) suppressed >85% at ratio of 20:1 and 50:1; and restimulated T_{REG}s after 6 days of culture (T_{REG} A+) suppressed 100% at E/T of 50:1.

### Example 8

### T_{REG} Suppression of Epstein-Barr Virus (EBV) Cell Line Producing Anti-IA2 or Anti-GAD65

An EBV-transformed B cell line derived from a subject with T1D known to have high titers to IA2 was tested. This model may simulate unregulated autoantibody synthesis that occurs in the dysregulated T1D immune system or other autoimmune states. Using the same B cell EliSpot assay, anti-IA2 IgM and anti-IA2 IgG secreting B cells were suppressed by >50% in 24-48-hour co-culture assays.

### Example 9

### Binding of T_{REG}s Armed with Anti-PD1×Anti-IA2 to Islets

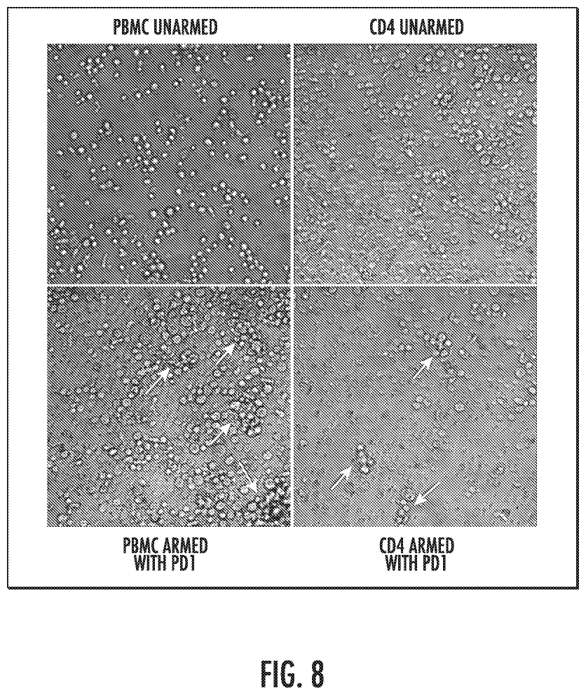

The specific approach is to target T_{REG}s to pancreatic beta cell sites that are expressing or leaking levels of IA2 or GAD65 that will allow T_{REG}s to bind to the islet cell. The strategy is to enhance trafficking and binding specific to the pancreatic islets. If enhanced numbers of specifically targeted T_{REG}s can be delivered to the inflammatory beta cell microenvironment, the T_{REG}s will have an opportunity to exert their suppressive activity locally. T_{REG}s armed with non-activating anti-T cell mAbs as partners to anti-IA2 or anti-GAD65 for targeting without losing suppressor function or inducing cytotoxic function at pancreatic islet cells/tissue. T_{REG}s armed with 500 ng of anti-PD1×anti-IA2 BiAb/10⁶ cells were mixed with freshly isolated islets at a ratio of 25:1 and incubated at 37° C. for 4 hours (FIG. 8).

### REFERENCES

All references listed herein above in the instant disclosure, and listed below, including but not limited to all patents, United States and PCT International patent applications and publications thereof, scientific journal articles, and database entries (including but not limited to Uniprot, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Lind et al: Glycemic control and excess mortality in type 1 diabetes. N Engl J Med. 371:1972-82. doi: 10.1056/NEJMoa1408214., 2014
2. Bluestone et al: Type 1 diabetes immunotherapy using polyclonal regulatory T cells. Sci Transl Med. 7:315ra189. doi:10.1126/scitranslmed.aad4134., 2015
3. Keymeulen et al: Four-year metabolic outcome of a randomised controlled CD3-antibody trial in recent-onset type 1 diabetic patients depends on their age and baseline residual beta cell mass. Diabetologia. 53:614-23. Epub 2010 Jan. 14., 2010
4. Gottlieb et al: Failure to preserve beta-cell function with mycophenolate mofetil and daclizumab combined therapy in patients with new-onset type 1 diabetes. Diabetes Care. 33:826-32. doi: 10.2337/dc09-1349. Epub 2010 Jan. 12., 2010
5. Herold et al: Anti-CD3 monoclonal antibody in new-onset type 1 diabetes mellitus. N Engl J Med 346:1692-8, 2002
6. Moran et al: Interleukin-1 antagonism in type 1 diabetes of recent onset: two multicentre, randomised, double-blind, placebo-controlled trials. Lancet. 381:10.1016/S0140-6736(13)60023-9. Epub 2013 Apr. 5 doi:10.1016/S0140-6736(13)60023-9., 2013
7. Orban et al: Co-stimulation modulation with abatacept in patients with recent-onset type 1 diabetes: a randomised, double-blind, placebo-controlled trial. Lancet. 378:412-9. doi: 10.1016/S0140-6736(11)60886-6. Epub 2011 Jun. 28., 2011
8. Pescovitz et al: Rituximab, B-lymphocyte depletion, and preservation of beta-cell function. N Engl J Med. 361:2143-52. doi: 10.1056/NEJMoa0904452., 2009
9. Mastrandrea et al: Etanercept Treatment in Children With New-Onset Type 1 Diabetes: Pilot randomized, placebo-controlled, double-blind study. Diabetes Care. 32:1244-9. Epub 2009 Apr. 14 doi:10.2337/dc09-0054., 2009
10. Long et al: Rapamycin/IL-2 combination therapy in patients with type 1 diabetes augments Tregs yet transiently impairs beta-cell function. Diabetes. 61:2340-8. doi: 10.2337/db12-0049. Epub 2012 Jun. 20, 2012
11. Jin & Lum: IgG anti-tetanus toxoid antibody production induced by Epstein-Barr virus from B cells of human marrow transplant recipients. Cell Immunol. 101:266-273, 1986
12. Lum & Culbertson: The induction and suppression of in vitro IgG anti-tetanus toxoid antibody synthesis by human lymphocytes stimulated with tetanus toxoid in the absence of in vivo booster immunizations. J. Immunol. 135:185-191, 1985
13. Lum: Immunotherapy with Activated T Cells after High Dose Chemotherapy and PBSCT for Breast Cancer, in Dicke K A, Keating A (eds). Charlottesville, N.Y., Carden Jennings, 2000, pp 95-105
14. Lum et al: Targeted T-cell Therapy in Stage IV Breast Cancer: A Phase I Clinical Trial. Clin Cancer Res 21:2305-14, 2015
15. Lum et al: Phase II Clinical Trial using Anti-CD3×Anti-HER2 Bispecific Antibody Armed Activated T Cells (HER2 BATs) for HER2 Negative (0-2+) Metastatic Breast Cancer [abstract], 2019 ASCO Annual Meeting. Chicago, Ill., 2019
16. Thakur et al: Immune T cells can transfer and boost anti-breast cancer immunity. OncoImmunology:1-11, 2018
17. Vaishampayan et al: Phase I Study of Anti-CD3×Anti-Her2 Bispecific Antibody in Metastatic Castrate Resistant Prostate Cancer Patients. Prostate Cancer 2015:285193, 2015
18. Lum et al: Phase I Dose Escalation of Activated T Cells (ATC) Armed with Anti-CD3×Anti-CD20 Bispecific Antibody (CD20Bi)&nbsp; after Stem Cell Transplant (SCT) In Non-Hodgkin's Lymphoma (NHL). The American Society of Hematology (ASH) December 4-7, 2010, Orlando, Fla.: Abstract #488, 2010
19. Lum et al: Multiple infusions of CD20-targeted T cells and low-dose IL-2 after SCT for high-risk non-Hodgkin's lymphoma: a pilot study. Bone Marrow Transplant 49:73-9, 2014
20. Lum et al: Targeting CD138−/CD20+ Clonogenic Myeloma Precursor Cells Decreases These Cells and Induces Transferable Antimyeloma Immunity. Biology of Blood and Marrow Transplantation 22:869-878, 2016
21. Yankelevich et al: Anti-CD3×anti-GD2 bispecific antibody redirects T-cell cytolytic activity to neuroblastoma targets. Pediatr Blood Cancer 59:1198-205, 2012
22. Lum L G, Thakur A, Kondadasula S V, et al. Targeting CD138-/CD20+ Clonogenic Myeloma Precursor Cells Decreases These Cells and Induces Transferable Antimyeloma Immunity. Biol Blood Marrow Transplant 2016; 22:869-78.
23. Rathore B, Davol P, Rathore R, et al. Trial of anti-CD3×anti-EGFR bispecific antibody (EGFRBi) in patients with EGFR-expressing recurrent/metastatic non-small cell lung cancer (NSCLC) and solid tumors. Journal of Clinical Oncology 2012; 30.
24. Lum L G, Thakur A, Al-Kadhimi Z, et al. Targeted T-cell Therapy in Stage IV Breast Cancer: A Phase I Clinical Trial. Clin Cancer Res 2015; 21:2305-14.
25. Lum L G, Le T, Choi M, et al. Exceptional Clinical and Immune Responses Using Anti-CD3×anti-EGFR Bispecific Antibody Armed T cells (BATs) for Locally Advanced or Metastatic Pancreatic Cancer [abstract]. 2019 ASCO Annual Meeting; 2019 May 31-Jun. 4; Chicago, Ill.
26. Lee R J, Fang Q, Davol P A, et al. Antibody targeting of stem cells to infarcted myocardium. Stem Cells 2007; 25:712-7.
27. Lum L G, Ramesh M, Thakur A, et al. Targeting cytomegalovirus-infected cells using T cells armed with anti-CD3×anti-CMV bispecific antibody. Biol Blood Marrow Transplant 2012; 18:1012-22.
28. Sen M, Wankowski D M, Garlie N K, et al. Use of anti-CD3×anti-HER2/neu bispecific antibody for redirecting cytotoxicity of activated T cells toward HER2/neu+ tumors. J Hematother Stem Cell Res 2001; 10:247-60.
29. Triponez F, Oberholzer J, Morel P, et al. Xenogeneic islet re-transplantation in mice triggers an accelerated, species-specific rejection. Immunology 2000; 101:548-54.
30. Avila J G, Wang Y, Barbaro B, et al. Improved outcomes in islet isolation and transplantation by the use of a novel hemoglobin-based O₂ carrier. Am J Transplant 2006; 6:2861-70.
31. Avila J, Barbaro B, Gangemi A, et al. Intra-ductal glutamine administration reduces oxidative injury during human pancreatic islet isolation. Am J Transplant 2005; 5:2830-7

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method of treating a disease and/or disorder associated with inflammation, the method comprising administering to the subject an effective amount of a composition comprising a targeted activated regulatory T cell (AT_{REG}), which selectively binds a cell associated with disease and/or disorder associated with inflammation in the subject, to thereby treat the disease and/or disorder associated with inflammation in the subject.

2. The method of claim 1, wherein the targeted activated regulatory T cell (AT_{REG}) is a bispecific antibody (BiAb) armed activated regulatory T cell (AT_{REG}).

3. The method of claim 1, wherein the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof.

4. The method of claim 1, wherein the method comprises:
(a) isolating peripheral blood mononuclear cells from a subject suffering from a disease and/or disorder associated with inflammation;
(b) arming a population of T regulatory cells (T_{REG}s) with a bispecific antibody (BiAb) directed at a T_{REG} cell and to target an antigen of a cell associated with the disease and/or disorder associated with inflammation in the subject, optionally pancreatic islet cells or other inflamed target cell, under conditions wherein:
(i) generation of T regulatory cells (T_{REG}s) occurs;
(ii) bispecific antibody are used to arm T_{REG} cells (AT_{REG}s) and target the cell having the antigen, optionally an autoimmune antigen (such as IA2, GAD65, ZNT8, pro-insulin, or other autoantigen) or an infectious agent antigen (such as a COVID19 antigen);
(iii) AT_{REG}s bind to the cell; and
(iv) suppression of inflammatory activity by immune cells in the subject by AT_{REG} occurs; and
(c) infusing a composition comprising the AT_{REG}s armed with a bispecific antibody into the subject, thereby treating the subject.

5. The method of claim 1, further comprising infusing AT_{REG}s intravenously and/or directly injecting AT_{REG}s into an affected organ and/or site with or without an additional therapeutic agent, optionally IL-2, immune suppressive cytokines, immunosuppressive agents, and/or immunosuppressive monoclonal antibodies.

6. The method of claim 1, wherein the AT_{REG}s have been induced by ex vivo stimulation with an anti-CD3 Mab/IL-2 and/or anti-CD3/anti-CD28 in combination with rapamycin and/or temsirolimus with TGF-beta, optionally in a range of 1 ng/ml to 200 ng/ml.

7. The method of claim 1, wherein the T_{REG}s have been induced and/or maintained by an ex vivo treatment or by an in vivo treatment of the subject with a checkpoint inhibitor antibody selected from the group consisting of anti-PD1 (CD279), anti-PDL1 (CD274 or B6 B7-H1), anti-PDL2 (CD273), anti-CTLA4 (CD152), or any combination thereof, wherein the checkpoint inhibitor antibody or antibodies enhance suppressor activity in the T_{REG}s.

8. The method of claim 1, wherein potency and phenotype of T_{REG}s can be induced, enhanced, and/or maintained by in vitro arming or in vivo arming the T_{REG}s with a BiAb with an anti-T cell partner being a checkpoint inhibitor agonistic to induce suppressor activity.

9. The method of claim 1, wherein the AT_{REG}s are from an autologous donor to the patient and/or are from an allogeneic donor to the patient.

10. The method of claim 1, wherein the T_{REG}s is a CD4⁺/FoxP3⁺ cell or a CD8⁺/FoxP3⁺ cell.

11. The method of claim 1, wherein arming doses provide 50% suppression at E:T of 1:1 to 5:1 in an immune suppression assay.

12. The method of claim 2, wherein the T_{REG}s can be armed with BiAbs doses ranging from 0.01 ng/million to 500 ng/million T_{REG}s.

13. The method of claim 2, wherein the BiAb comprises two monoclonal antibodies.

14. The method of claim 2, wherein the BiAb is directed at any non-activating T cell antigen.

15. The method of claim 1, wherein the AT_{REG} is targeted at any surface antigen on pancreatic islet or organ cell being damaged by an inflammatory process, optionally IA2, GAD65, or ZNT8, or is targeted at a COVID 19 antigen, optionally a SAR-CoV2 antigen, further optionally spike, S1 receptor binding domain, nucleiocapside, or membrane antigen.

16. A composition comprising an effective amount of a targeted activated regulatory T cell (AT_{REG}), which selectively binds a cell associated with disease and/or disorder associated with inflammation in the subject; and a pharmaceutically acceptable carrier.

17. The composition of claim 16, wherein the targeted activated regulatory T cell (AT_{REG}) is a bispecific antibody (BiAb) armed activated regulatory T cell (AT_{REG}).

18. The composition of claim 16, wherein the disease and/or disorder associated with inflammation comprises an autoimmune disease, optionally Type 1 diabetes, a graft-vs-host disease, an organ graft rejection, an infection, optionally a COVID19 infection, inflammatory acute respiratory distress syndrome, and/or any combination thereof.

19. The composition of claim 16, for use in treating a disease and/or disorder associated with inflammation in the subject.

20-22. (canceled)

23. A composition for treating a mammal suffering from autoimmune diseases, graft-vs-host diseases, and/or organ graft rejection (collectively AGO) comprising AT_{REG}s targeting checkpoint antigens on the mammal's T cells and autoimmune antigen targets in inflamed tissue of the mammal.
